(19) United States
(12) Patent Application Publication
(10) Pub. No.: US 2007/0048813 A1
(43) Pub. Date: Mar. 1, 2007

(54) Agent for assaying analyte of patient by enzyme

(75) Inventors: Xiong Chen, Shanghai city (CN); Wang-Ge Liang, Shanghai city (CN)

Correspondence Address: BIRCH STEWART KOLASCH & BIRCH, PO BOX 747, FALLS CHURCH, VA, 22040-0747, US

(73) Assignee: FENGHUI (SHANGHAI) MEDICAL SCIENCE& TECH. CO., LTD, SHANGHAI, CHINA (CN)

(21) Appl. No.: 10/574,643

(22) PCT Filed: Sep. 5, 2003
(86) PCT No.: PCT/CN03/00749

§ 371 Date: Apr. 4, 2006

(51) Int. Cl. C12Q 1/54; C12Q 1/48

(52) U.S. Cl. 435/14; 435/15

(57) ABSTRACT

This invention concerns an enzymatic reagent for measuring the analyte concentration in a patient by determination of oxidation rate of a reduced coenzyme. Said reagent is stabilized by the coenzyme reduction system which makes the reduced coenzyme in the reagent regenerate continuously throughout a long storage. This coenzyme reduction system comprises an enzyme having high specificity for said substrate, which results that the quantity of enzyme and substrate originally used in the reagent is reduced and the stability of reagent is improved. The reagent is a sole liquid. An essential part of the invention is a reagent to determine the anlytes for example aspartate aminotransfase, alanine aminotransferase and urea.

### TECHNICAL FIELD

This invention involves a reagent for enzymatic determination of an analyte concentration in a patient, especially involves the reagents which measures the degree of oxidation of reduced coenzyme which quantity corresponds directly to the concentration of analyte present in the sample.

### TECHNOLOGY BACKGROUND

In clinic, analytes that can be measured by determining the degree of oxidation of β-NADH include aspartate aminotransferase, alanine aminotransferase, ammonia, urea, lactate dehydrogenase, carbon dioxide and α-hydroxyl butyric acid dehydrogenase.

Aspartate aminotransferase is widely distributed in human body especially higher in heart, liver, kidney, red blood cells and skeletal muscle. Increases of levels of aspartate aminotransferase in serum are found in tissue destruction such as myocardial infarction, liver cell destruction, hepatitis, hepatocirrhosis, malnutrition.

When the activity of aspartate aminotransferase(AST) is determined AST in the serum catalyses amino transformation from α-ketoglutarate to L-aspartate to form L-glutamic acid and oxaloacetate. In the presence of reduced coenzyme I (β-NADH) and malate dehydrogenase (MDH), oxaloacetate is converted to malate. This is accompanied by the oxidation of the coenzyme nicotinamide adenine dinucleotide (β-NADH to β-NAD⁺) which can lower the absorbance at 340 nm. Thus the reaction sequence is commonly as follows:

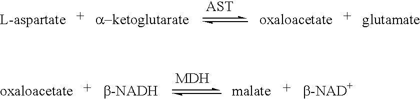

Lactate dehydrogenase that exists in serum can convert intrinsic pyruvate to lactic acid and oxidize β-NADH, as a result it interferes with determination. High levels of lactate dehydrogenase can quickly eliminated this side reaction in the lag phase. The reaction is as follows: 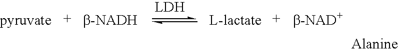
aminotransferase (ALT) is existed in high concentration in the liver but low levels in heart, kidney, lung and skeletal muscle. Usually increasement in the level of ALT in the serum is concerned with some liver diseases including hepatocirrhosis, liver cancer, hepatitis, obstructive and icterus.

When the activity of alanine aminotransferase (ALT) is determined ALT in the serum catalyse amino transformation from L-alanine to α-oxoglutarate to form L-glutamate and pyruvate. In the presence of reduced coenzyme I (β-NADH) and lactate dehydrogenase (LDH), pyruvate is converted to L-lactate. This is accompanied by the oxidation of the coenzyme nicotinamide adenine dinucleotide (β-NADH to β-NAD⁺) which can lower absorbance at 340 nm. Thus the reaction sequence is as follows:

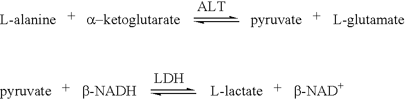

The interference by intrinsic pyruvate in serum can be eliminated through adding excessive lactate dehydrogenase. The reaction is as follows:

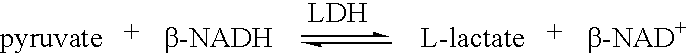

Urea is the major nitrogen-containing metabolic product from protein catabolism, being formed in the liver and excreted through the kidneys. Elevated levels of urea in serum may be a consequence of impaired kidney function and urethra block. Hence the level of urea in blood is an important sign of kidney function.

When the concentration of urea is determined urea decomposes to ammonia and carbon dioxide in catalysis by urease. The ammonia and α-ketoglutarate is converted to glutamate in the presence of β-NADH and glutamate dehydrogenase (GLDH). Simultaneously β-NADH is oxidized to β-NAD⁺ which can lower the absorbance at 340 nm. So the concentration of urea can be determinated by spectrophotometric method. The reactions are as follows:

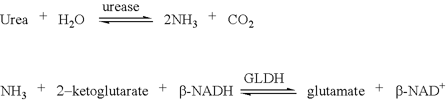

The interference by intrinsic ammonium of serum can be quickly eliminated in the delay lag. The reaction is as follows: 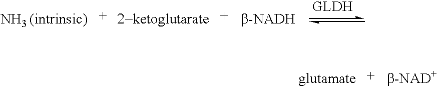
In order to stabilize the assay reigns for long including AST, ALT and UREA in a single vial format, it is important to resolve the stability of βNADH and tool enzymes. Since various enzymes are precisely constructed protein which show poor stability, many factors including temperature, pH, ion strength, impurities, metal ions and microorganisms all can affect their activity. To improve the enzyme stability in aqueous solution, it is feasible to improve the surroundings of the enzymes including addition of preservatives and stabilizers and so on. Tool enzymes should be selected from enzymes which show high thermostability, less impurities, and a good stability in the pH range of determination. The quantity of tool enzymes should be appropriate in order to ensure the exactness of the assay result and the tool enzyme can stabilize for a long time.

The difficulty to assure reagent stability mainly lies in the stability of β-NADH which is the common indicator for the assay reagents: AST, ALT and UREA. In order to guarantee the proper linearity in the assay, β-NADH in the reagent should maintain in a suitable concentration, namely the absorbance at 340 nm can not be lower than 1.0 A. But β-NADH in aqueous solution at pH<8.6 is unstable and can spontaneously be oxidized to β-NAD⁺, and can be catalized to β-NAD⁺ by other enzymes in the solution.

In order to increase the stability of β-NADH, some people had made massive research works in 1970's. They utilized general physical methods, such as freezing and drying the reagent into powder, or increased the NADH stability with some anhydrous organic solvents. In 1977, Modrovich (U.S. Pat. No. 4,394,449) stated that Glucose-6-Phosphate dehydrogenase(G-6-PDH)/Glucose-6-Phosphate(G-6-P) pair can revert the product β-NAD⁺ to β-NADH, and stabilize β-NADH in the reagent The reaction is as follows: 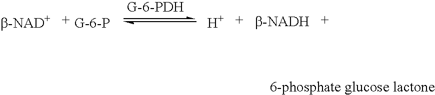
At that time the development of enzyme engineering didn't as good as today, the reagents only lied in two vials because the key technology did not be resolved. In case the reagents made into a single vial, the storage life was only between one to three months. In 1990's F Hoffman la Roche AG(AU-A-61906/90) had done much work based on Modrovich's principle. But his method can only prepare the double reagent, once prepared in the single reagent, the stability is poor. Klose et al (in U.S. Pat. No. 4,019,916) put forward a similar Method, but that took a long time to test and it was only suitable for testing system in which there is a substrate which can be phosphated. De Giorgio et al (Australia) in Feb. 26, 1996 applied their patent in China (CN1179792A). In that patent non-specific enzyme/substrate pair was successfully used in single reagent (AST, ALT) and two reagent (UREA), based on dynamic stabilization technology. The shelf life of the single liquid reagent (AST, ALT) was extended to 6-8 months. Although De Giorgio et al made improvement on predecessor's foundation, in the patent he claimed that the enzyme had incomplete specificity to the substrate and the enzyme/substrate pair was limited to glucose-6-phosphate dehydrogenase/D-glucose. The quantities of glucose-6-phosphate dehydrogenase/D-glucose are very big glucose-6-phosphate dehydrogenase 3500 U/L, D-glucose 18.016 g/L. This not only obviously increases the cost, but also raises the possibility of introducing other enzymes in it.

### INVNETION DESCRIPTION

In view of the existing technical insufficiency of the tests, in this invention we claim an enzymatic method for determination of analyte concentration in patient. Said reagent relates to determine the oxidation rate of reduced coenzyme. It certainly not obviously increases the cost, but can prevent other enzymes introduction, and has a long-term stability.

Said reagent is stabilized throughout storage by coenzyme reduction system of special enzyme/substrate pair in which coenzyme can be regenerated. Said enzyme is highly special for said substrate in the enzyme/substrate pair.

Said reagent is configured as a single vial in liquid; glucose dehydrogenase/D-glucose pairs is prior to other enzyme/substrate pairs in coenzyme reduction system.

The invention also describes the enzyme reagent for determining the concentration of aspartate aminotransferase. The oxidation rate of reduced coenzyme is determined in the assay. Said reagent in a single vial is stabilized by the regeneration of reduction coenzyme at storage life based on coenzyme reduced system comprising special enzyme/substrate pair wherein said enzyme is special for said substrate. Glucose dehydrogenase/D-glucose pair is prior to other enzyme/substrate pairs. The concentration of said glucose dehydrogenase is in the range of 2-100 U/L (5-50 U/L is optimal) and D-Glucose is in the range of 0.1-20 mmol/L (1-10 mmol/L is optimal).

The invention also describes the enzyme reagent for determining the concentration of alanine aminotransferase. The oxidation rate of reduced coenzyme is determined at the test. Said reagent in a single vial is stabilized by the regeneration of reduced coenzyme at storage life based on coenzyme reduction system comprising special enzyme/substrate pair wherein said enzyme is special for said substrate. Glucose dehydrogenase/D-glucose pair is prior to other enzyme/substrate pairs. The concentration of said glucose dehydrogenase is in the range of 2-100 U/L (5-50 U/L is optimal) and D-glucose is in the range of 0.1-20 mmol/L (1-10 mmol/L is optimal).

The invention also describes the enzyme reagent for determining the concentration of urea. The oxidation rate of reduced coenzyme is determined at the test. Said reagent in a single vial is stabilized by the regeneration of reduced coenzyme at storage life based on coenzyme reduction system comprising special enzyme/substrate pair wherein said enzyme is special for said substrate. Glucose dehydrogenase/D-glucose pair is prior to other enzyme/substrate pairs. The concentration of said glucose dehydrogenase is in the range of 2-100 U/L (5-50 U/L is optimal) and D-glucose is in the range of 0.1-20 mmol/L (1-10 mmol/L is optimal).

In the regeneration system of β-NADH comprising dehydrogenase/substrate pair, glucose dehydrogenase is completely special for D-glucose. D-glucose is converted to D-glucose lactone accompanying the reduction of β-NAD⁺ to β-NADH. The reaction is as follows: 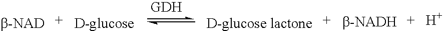
Glucose dehydrogenase is stable at pH 6-8.5 in test, so the reagent is stable in test at pH 7.2-8.5. However the optimum pH 8.0 of glucose dehydrogenase is in the range of pH 7.2-8.5. Because the enzyme is in this circumstance wherein enzyme reaction rate is fast and enzyme is in the prior pH, the quantity of dehydrogenase and substrate is highly reduced. As a result, not only the reagent stability is improved because of avoiding contamination other enzymes but also the cost falls down.

The rate of regenerated β-NADH is controlled by modulating the quantity of glucose dehydrogenase and glucose in the reagent. In general, the rate of β-NADH regeneration is same as the rate of β-NADH oxidation. The coenzyme can be regenerated in coenzyme reduction system of regeneration, which has no effect on the assay.

In the β-NADH regeneration systems the concentration of glucose dehydrogenase is in the range of 2-100 U/L and glucose is in the range of 0.1-20 mmol/L. Higher concentration of glucose dehydrogenase or glucose will result that the rate of regeneration β-NADH is too fast. And the negative interference will come out in the assay.

As said in this invention that reagent used to determine AST in the sample comprises not only coenzyme reduction system including glucose dehydrogenase/D-glucose but also malate dehydrogenase(MDH), lactate dehydrogenase, reduced coenzyme I(β-NADH), L-aspartate and 2-ketoglutarate.

Reagents in this invention used to detect ALT in the sample comprises not only glucose dehydrogenase/D-glucose as coenzyme reduction system but also lactate dehydrogenase(LDH), reduced coenzyme I (β-NADH) and 2-ketoglutarate and so on.

Reagents in this invention used to determine the concentration of urea in the sample comprise not only glucose dehydrogenase/D-glucose as coenzyme reduced system but also urease, glutamate dehydrogenase (GLDH), reduced coenzyme I (β-NADH) and 2-ketoglutarate.

Reagents in the invention comprise not only essential coenzyme reduction system, basic substrate and enzyme but also the buffer, preservative, stabilizer and chelator and so on, and more substances which can strengthen the stability but not affect the determination.

Glycerine, sugar and glycol are the polyhydroxylated compounds, which can form many hydrogen bonds with the protein molecules, which is helpful to the formation of ‘solvent layer’. The solvent layer around the enzyme molecules is different from the overall aqueous phase because it can improve the surface tension and solution viscosity. This kind chemical additive through effective dehydration can reduce the hydrolysis of the protein and therefore stabilize the enzyme. The enzyme can be stabilized by using the relatively low molecular weight polyols. We select glyceroland glycol as the stabilizers. But too high concentration is adverse to the detection because of the high solution viscosity.

EDTA disodium and heavy metal ions can form coordinate compound to avoid enzyme being inhibited by the heavy metal ion.

The microorganism pollution can reduce the stability of enzyme, addition of antiseptic may suppress the microorganism growth. In this invention, azide sodium is prior to other preservatives.

In this invention liquid reagents based on stabilization technology of coenzyme applying glucose dehydrogenase/D-glucose pair are configured as a single vial. And said reagents to determine AST mainly comprise coenzyme reduction system (glucose dehydrogenase/D-glucose), 1-aspartate, α-oxoglutarate, malate dehydrogenase (MDH), lactate dehydrogenase, reduced coenzyme I (β-NADH).

In addition, prior reagents selected comprise Tris-HCl buffer, potassium hydroxide, EDTA disodium salt, glycerol, sodium azide.

The concentration of tris-HCl buffer is in the range of 20-100 mmol/L; The concentration of α-oxoglutarate is limited in the range of 6-18 mmol/L because of it shows absorbency at 340 nm; the concentration of L-aspartate is in the range of 100-300 mmol/L; The concentration of potassium hydroxide is the same as L-aspartate to increase the solubility of L-aspartate; The concentration of EDTA disodium is in the range of 1-10 mmol/L in order to prevent the suppression of heavy metal ions to enzyme activity through formation coordinate compound with heavy metal ions; The concentration of β-NADH is in the range of 0.1-0.3 mmol/L. When the concentration of β-NADH is lower than 0.1 mmol/L the assay result is affected since the linear scope range being shortened while the concentration of β-NADH is more than 0.3 mmol/L the blank absorbency is too high for assay; The concentration of malate dehydrogenase (MDH) is in the range of 100-2500 U/L; The addition of lactate dehydrogenase in the range of 1000-4000 U/L is to eliminate the interference from pyruvate in the sample; The addition of glucose dehydrogenase/D-glucose in the range of 2-100 U/L aims at the regeneration of β-NADH from it's oxidation product (NAD) so as to assure the stabilization of β-NADH; The concentration of D-glucose is in the range of 0.1-20 mmol/L; The amount of glycerol is 1%-20% to make enzymes more stable. Higher concentration of glycerol will increase the viscosity of solution. The concentration of sodium azide is in the range of 0.1-1.0 g/L to prevent the microorganism pollution.

According to the invention one kind of optimal reagent to determine AST comprises the ingredients being listed in the following table:

TABLE 1

| | Molecular | concentration | |
|---|---|---|---|
| ingredients | weight | (mmol/L) | /L |
| tris | 121.1 | | |
| sodium α-oxoglutarate, | 226.1 | | |
| (2H₂O) | | | |
| L-aspartate | 133.1 | | |
| potassium hydroxide | 56.1 | | |
| D-glucose | 180.2 | | |
| glycerol | 92.1 | | |
| EDTA.2Na | 372.2 | | |
| sodium azide | 65.1 | | |
| β-NADH disodium | 709.4 | | |
| lactate dehydrogenase | | | |
| malate dehydrogenase | | | |
| glucose dehydrogenase | | | |
| hydrochloric acid | 36.5 | | |

In this invention liquid reagents based on stabilization technology of coenzyme applying glucosedehydrogenase/D-glucose are configured as a single vial. And said reagents to determine ALT mainly comprise coenzyme reduction system (glucose dehydrogenase/D-glucose), L-alanine, α-oxoglutarate, lactate dehydrogenase, reduced coenzyme I (β-NADH). In addition the prior reagents comprise Tris-HCl buffer, EDTA disodium, glycerol and sodium azide. The concentration of Tris-HCl buffer is prior in the range of 20-100 mmol/L; The concentration of α-oxoglutarate is in the range of 8-18 mmol/L; The concentration of L-alanine is in the range of 200-800 mmol/L; The concentration of EDTA disodium is in the range of 1-10 mmol/L; The concentration of β-NADH is in the range of 0.1-0.3 mmol/L; lactate dehydrogenase added in the range of 1000-400 U/L is used to eliminate the interference from pyruvate in the sample and to assure catalyzing reaction in the linear range The concentration of glucose dehydrogenase is in the range of 2-100 U/L and D-glucose is 0.1-20 mmol/L. The concentration of sodium azide is in the range of 0.1-100.0 g/L. The concentration of glycerol is in the range of 1%-20%.

According to this invention one kind of optimal reagents to determine ALT comprises the ingredients being listed in the following table:

TABLE 2

| | | concentration | Amount |
|---|---|---|---|
| ingredients | Molecular weight | (mmol/L) | (g/L) |
| tris | 121.1 | | |
| sodium α-ketoglutarate | 226.1 | | |
| 2H₂O) | | | |
| L-alanine | 89.1 | | |
| D-glucose | 180.2 | | |
| EDTA2Na | 372.2 | | |
| glycerol | 92.1 | | |
| sodium azide | 65.1 | | |
| β-NADH disodium | 709.4 | | |
| lactate dehydrogenase | | | |
| glucose dehydrogenase | | | |
| hydrochloric acid | 36.5 | | |

In this invention liquid reagents based on stabilization technology of coenzyme applying glucose dehydrogenase/D-glucose are configured as a single vial. And said reagents to determine urea mainly comprise coenzyme reduction system (glucose dehydrogenase/D-glucose), α-oxoglutarate, urease, glutamate dehydrogenase(GLDH), reduced coenzyme I(β-NADH).

In addition the optimal reagents comprise tris-HCl buffer, potassium adenosine diphosphate, glycerol and sodium azide. The concentration of Tris-HCl buffer is in the range of 20-150 mmol/L; The concentration of α-oxoglutarate is in the range of 1-15 mmol/L; The concentration of β-NADH is in the range of 0.10-0.38 mmol/L; The concentration of potassium ADP is in the range of 0.1-10.0 mmol/L; The concentration of urease is in the range of 2000-1000 U/L for fast catalyzing decomposition of urea; Addition of glutamate dehydrogenase can control the reaction rate, more higher concentration will make the reaction rate quicker. The optimal concentration of glutamate dehydrogenase is in the range of 200-2000 U/L; The concentration of Glucose dehydrogenase is in the range of 2-100 U/L; The concentration of D-glucose is in the range of 0.1-20 mmol/L; The concentration of sodium azide is in the range of 0.1-1.0 g/L; The concentration of glycerol is in the range of 1%-30%.

According to this invention one kind of optimal reagent to determine UREA includes ingredients being listed in the following table:

TABLE 3

| | | Concentration | |
|---|---|---|---|
| ingredients | Molecular weight | (mmol/L) | /L |
| tris | 121.1 | | |
| α-ketoglutarate | 226.1 | | |
| sodium azide | 65.1 | | |
| D-glucose | 180.2 | | |
| glycerol | 92.1 | | |
| ADP.2K | 501.3 | | |
| β-NADH disodium | 709.4 | | |
| GLDH | | | |
| urease | 709.4 | | |
| glucose dehydrogenase | | | |
| hydrochloric acid | 36.5 | | |

In the liquid single reagent, the selected Lactate dehydrogenase should have higher affinity with pyruvic acid, and contain micro absent from other enzyme such as ALT, GLDH and so on. The selected malic dehydrogenase and glutamic dehydrogenase should be more stable in aqueous solution. On the premise of keeping test linearity, delay time, accuracy and stability, the amount of the above enzymes should be reduced as far as possible, in order to eliminate the interference of other enzyme.

In addition to AST, ALT and UREA, the analytes which can be determined by the reagents of the invention include ammonia, lactate dehydrogenase, carbon dioxide, α-hydroxyl butyrate dehydrogenase and so on.

Furthermore, β-NADPH can be regenerated from NADP produced by oxidation in the case of glucose dehydrogenase/D-glucose.
The reaction is as follows:

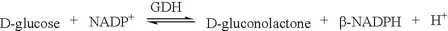

In contrast to the existing technology, the merit of this invention is as follows: the quantity of enzyme and substrate is reduced by the introduction of coenzyme reduction system against oxidation comprising highly specific enzyme/substrate pair. As a result, the other enzymes are excluded. So the cost of the reagent is reduced, but the stability is improved.

### PREFERRED EMBODIMENTS

The details of this invention are clearly described by the following preferred examples.

### Example 1

Determination of the stability of AST reagent (D-glucose: 5 mmol/L, glucose dehydrogenase: 20 U/L) formulated in accordance with the invention is as follows.

One kind of stabilized AST reagent in a single vial is as follows:

TABLE 4

| | Molecular | Concentration | |
|---|---|---|---|
| ingredients | Weight | (mmol/L) | /L |
| tris | 121.1 | | |
| α-ketoglutarate, | 226.1 | | |
| (2H₂O) | | | |
| L-aspartate | 133.1 | | |
| potassium hydroxide | 56.1 | | |
| D-glucose | 180.2 | | |
| glycerol | 92.1 | | |
| EDTA.2Na | 372.2 | | |
| sodium azide | 65.1 | | |
| β-NADH disodium | 709.4 | | |
| lactate dehydrogenase | | | |
| malate dehydrogenase | | | |
| glucose dehydrogenase | | | |
| hydrochloric acid | 36.5 | | |

The correspondent unstabilized liquid reagent configured as a single vial does not include the coenzyme reduction system of Glucose dehydrogenase/D-glucose and absent from glycerol, but comprises the ingredients as above stable.

Storage Conditions:

Sealed up and stored at 2-8° C. or 37° C.

Spectrophotometric Parameters:

| | |
|---|---|
| wavelength: 340 nm | reaction temperature: 37° C. |
| cuvette path length: 10 mm | |
| volume ratio of sample to reagent: 1:15 | |
| lag phase: 60 seconds | reaction time: 60 seconds |
| blank absorbance of reagent: >1.0 A | |
| (for showing the concentration of NADH) | |
| assay precision: the result should be | |
| in the range of the quality control serum. | |
| linearity range: ≧550 U/L | |
| assayed results: | |

1) The initial absorbance of AST reagent stored at 37° C.

TABLE 5

| storage at | Stabilized | Unstabilized |
|---|---|---|
| 37° C. (days) | reagent | reagent |
| 0 | 1.788 | 1.809 |
| 1 | 1.662 | |
| 2 | 1.538 | |
| 3 | 1.415 | 1.019 |
| 4 | 1.313 | |
| 5 | 1.188 | |
| 6 | 1.148 | |
| 7 | 1.043 | |

It is obvious that stabilized AST reagent in a single vial has a storage of seven days at 37° C. But the unstabilized AST reagent has only a storage of three days at 37° C. β-NADH in the stabilized reagent is more stable than others.

2) The initial absorbance of AST reagent stored at 2-8° C.

TABLE 6

| Storage at | | Stabilized | Unstabilized |
|---|---|---|---|
| 2-8° C. | | reagent | reagent |
| 0 | week | 1.839 | 1.809 |
| 3 | weeks | | 1.452 |
| 5 | weeks | | 1.319 |
| 7 | weeks | | 1.210 |
| 11 | weeks | | 1.023 |
| 3 | months | 1.665 | |
| 6 | months | 1.477 | |
| 9 | months | 1.305 | |
| 12 | months | 1.102 | |

β-NADH in the stabilized AST reagent in a single has a storage more than 12 months at 2-8° C. But β-NADH in the unstabilized AST reagent has a storage of 11 weeks at 2-8° C.

3) linearity assay of stabilized AST reagent in a single vial stored at 2-8° C.

TABLE 7
stored at 2-8° C. for three months

| | | | | | | |
|---|---|---|---|---|---|---|
| theory value | 0 | 116 | 233 | 349 | 466 | 582 |
| U/L | | | | | | |
| assay value U/L | 4.8 | 111 | 227 | 349 | 452 | 585 |

stored at 2-8° C. for six months

| | | | | | | |
|---|---|---|---|---|---|---|
| theory value U/L | 0 | 113 | 226 | 338 | 451 | 564 |
| assay value U/L | 5.1 | 115 | 220 | 338 | 436 | 559 |

stored at 2-8° C. for nine months

| | | | | | | |
|---|---|---|---|---|---|---|
| theory value U/L | 0 | 105 | 210 | 315 | 420 | 524 |
| assay value | 4.9 | 106 | 215 | 315 | 406 | 517 |
| U/L | | | | | | |

stored at 2-8° C. for thirteen months

| | | | | | | |
|---|---|---|---|---|---|---|
| theory value | 0 | 122 | 244 | 366 | 488 | 610 |
| U/L | | | | | | |
| assay value | 5.5 | 124 | 247 | 366 | 473 | 582 |
| U/L | | | | | | |

The result of linearity assay of the stabilized AST reagent in a single vial is up to the mustard after being stored at 2-8° C. for thirteen months.

4) accuracy assay of stabilized AST reagent in a single vial stored at 37° C.

TABLE 8

| | Serum I (U/L) | Serum II (U/L) | Serum III (U/L) |
|---|---|---|---|
| Storage at 37° C. | target value 30 | target value 54 | target value |
| (days) | (20-40) | (41-67) | 101 (81-121) |
| 0 | 28 | 54 | 98 |
| 5 | 28 | 56 | 92 |
| 6 | 27 | 52 | 95 |
| 7 | 26 | 53 | 95 |

The results of accuracy assay of the stabilized AST reagent in a single vial accord with the target values of the quality-controlled serum after being stored at 37° C. for seven days.

5) Accuracy assay of stabilized AST reagent stored at 2-8° C.

TABLE 9

| storage at | | |
|---|---|---|
| 2-8° C. | | |
| (months) | Serum I (U/L) | Serum III (U/L) |
| 3 | target value: 28(23-33) | target value: |
| | assay value: 32 | 104(84-124) |
| | | assay value: 117 |
| 6 | target value: 28(23-33) | target value: 104(84-124) |
| | assay value: 32 | assay value: 101 |
| 9 | target value: | target value: 104(84-124) |
| | 28(23-33) | assay value: 110 |
| | assay value: 30 | |
| 12 | target value: | target value: |
| | 30(20-40) | 101(81-121) |
| | assay value: 32 | assay value: 106 |

The result of accuracy assay of the stabilized AST reagent in a single vial are all within the scope of the target value of the quality-controlled serum after being stored at 2-8° C. for 12 months.

The above results showed that the assay data of the stabilized AST reagent in a single vial was up to the mustard after being stored at 2-8° C. for twelve months or at 37° C. for seven days. In short, the method of utilizing coenzyme (NADH) reduced system of glucose dehydrogenase/D-glucose pair is feasible.

### Example 2

Determination of the stability of AST reagent (D-glucose: 1 mmol/L, glucose dehydrogenase: 5 U/L) formulated in accordance with the invention is as follows.

One stabilized AST liquid reagent in a single vial is as follows:

TABLE 10

| | Molecular | Concentration | |
|---|---|---|---|
| ingredients | Weight | (mmol/L) | /L |
| Tris | 121.1 | | |
| α-ketoglutarate (2H₂O) | 226.1 | | |
| L-aspartate | 133.1 | | |
| potassium hydroxide | 56.1 | | |
| D-glucose | 180.2 | | |
| glycerol | 92.1 | | |
| EDTA.2Na | 372.2 | | |
| sodium azide | 65.1 | | |
| β-NADH disodium | 709.4 | | |
| Lactate dehydrogenase | | | |
| malate dehydrogenase | | | |
| glucose dehydrogenase | | | |
| hydrochloric acid | 36.5 | | |

A correspondent unstabilized liquid reagent configured as a single vial does not include the coenzyme reduction system of glucose dehydrogenase/D-glucose and absent from glycerol, but comprises other ingredients as described in stable 10.

Storage Conditions:

Seal up and stored at 2-8° C. or 37° C.

Spectrophotometric Parameters:

| | |
|---|---|
| wavelength: 340 nm | reaction temperature: 37° C. |
| cuvette path length: 10 mm | |
| volume ratio of sample to reagent: 1:15 | |
| lag phase: 60 seconds | reaction time: 60 seconds |
| bank absorbance of reagent: >1.0 A | |
| (for showing the concentration of NADH) | |
| assay precision: the result should be in the | |
| range of the quality controlled serum. | |
| linearity range: ≧550 U/L | |
| assay results: | |

2) The blank absorbance of AST liquid reagent stored at 37° C.

TABLE 11

| Storage at 37° C. (days) | Stabilized reagent | Unstabilized reagent |
|---|---|---|
| 0 | 1.755 | 1.745 |
| 1 | 1.624 | |
| 2 | 1.490 | |
| 3 | 1.359 | 0.943 |
| 4 | 1.245 | |
| 5 | 1.110 | |

2) The blank absorbance of AST reagent in a single vial stored at 2-8° C.

TABLE 12

| Storage at | | Stabilized | |
|---|---|---|---|
| 2-8° C. | | reagent | Unstabilized reagent |
| 0 | week | 1.758 | 1.751 |
| 3 | weeks | | 1.396 |
| 5 | weeks | | 1.258 |
| 7 | weeks | | 1.150 |
| 11 | weeks | | 0.965 |
| 3 | months | 1.579 | |
| 6 | months | 1.385 | |
| 9 | months | 1.208 | |
| 12 | months | 0.998 | |

3) linearity assay of stabilized AST reagent in a single vial stored at 2-8° C.

TABLE 13
stored at 2-8° C. for three months

| | | | | | | |
|---|---|---|---|---|---|---|
| theory value | 31 | 125 | 250 | 374 | 499 | 624 |
| U/L | | | | | | |
| assay value U/L | 36 | 127 | 250 | 365 | 497 | 606 |

stored at 2-8° C. for six months

| | | | | | | |
|---|---|---|---|---|---|---|
| theory value U/L | 30 | 122 | 243 | 365 | 486 | 608 |
| assay value U/L | 33 | 122 | 258 | 365 | 483 | 578 |

stored at 2-8° C. for nine months

| | | | | | | |
|---|---|---|---|---|---|---|
| theory value U/L | 30 | 110 | 220 | 330 | 440 | 550 |
| assay value | 35 | 118 | 229 | 325 | 436 | 538 |
| U/L | | | | | | |

4) accuracy assay of stabilized AST reagent in a single vial stored at 37° C.

TABLE 14

| | Serum I (U/L) | Serum II (U/L) | Serum III (U/L) |
|---|---|---|---|
| storage at 37° C. | Target value 30 | Target value 54 | Target value |
| (days) | (20-40) | (41-67) | 101 (81-121) |
| 0 | 33 | 56 | 107 |
| 5 | 35 | 58 | 105 |

5) accuracy assay of stabilized AST reagent in a single vial stored at 2-8° C.

TABLE 15

| storage at | | |
|---|---|---|
| 2-8° C. | | |
| (months) | Serum I (U/L) | Serum III (U/L) |
| 3 | target value: 30(20-40) | target value: |
| | assay value: 36 | 101(81-121) |
| | | assay value: 115 |
| 6 | Target value: 30(20-40) | target value: 101(81-121) |
| | assay value: 35 | assay value: 108 |
| 9 | Target value: | target value: 101(81-121) |
| | 30(20-40) | assay value: 106 |
| | assay value: 32 | |

The above results showed that the assay data of the stabilized AST reagent are up to the mustard after being stored at 2-8° C. for 9 months or at 37° C. for 5 days. In short, the method of utilizing coenzyme (NADH) reduction system of glucose dehydrogenase/D-glucose pair is feasible.

### Example 3

Determination of the stability of AST reagent (D-glucose: 10 mmol/L, glucose dehydrogenase: 50 U/L) formulated in accordance with the invention is as follows.

One stabilized AST liquid reagent in a single vial is as follows:

Stable 16

| | Molecular | Concentration | |
|---|---|---|---|
| ingredients | Weight | (mmol/L) | /L |
| tris | 121.1 | | |
| Sodium α- | 226.1 | | |
| oxoglutarate, (2H₂O) | | | |
| L-aspartate | 133.1 | | |
| potassium hydroxide | 56.1 | | |
| D-glucose | 180.2 | | |
| glycerol | 92.1 | | |
| EDTA.2Na | 372.2 | | |
| sodium azide | 65.1 | | |
| β-NADH disodium | 709.4 | | |
| lactate dehydrogenase | | | |
| malate dehydrogenase | | | |
| glucose dehydrogenase | | | |
| hydrochloric acid | 36.5 | | |

The correspondence unstabilized liquid reagent configured as a single vial does not include the coenzyme reduction system of glucose dehydrogenase/D-glucose and absent from glycerol, but comprises the other ingredients as described in stable 16.

Storage Conditions:

Sealed up and stored at 2-8° C. or 37° C.

Spectrophotometric Parameters:

| | |
|---|---|
| wavelength: 340 nm | reaction temperature: 37° C. |
| cuvette path length: 10 mm | sample to reagent volume: 1:15 |
| lag phase: 60 seconds | reaction time: 60 seconds |
| initial absorbance of reagent: >1.0 A | |
| (to determine the concentration of | |
| NADH) assay precision: assay result | |
| should be in the range of the quality | |
| control serum. | |
| Linearity range: ≧550 U/L | |
| assay result: | |

3) The blank absorbance of AST liquid reagent in a single vial stored at 37° C.

TABLE 17

| Storage at 37° C. (days) | Stabilized reagent | Unstabilized reagent |
|---|---|---|
| 0 | 1.880 | 1.886 |
| 1 | 1.759 | |
| 2 | 1.640 | |
| 3 | 1.518 | 1.075 |
| 4 | 1.396 | |
| 5 | 1.285 | |
| 6 | 1.177 | |
| 7 | 1.080 | |

2) The blank absorbance of AST liquid reagent stored at 2-8° C.

TABLE 18

| Storage at | | Stabilized | |
|---|---|---|---|
| 2-8° C. | | reagent | Unstabilized reagent |
| 0 | week | 1.878 | 1.883 |
| 3 | weeks | | 1.530 |
| 5 | weeks | | 1.395 |
| 7 | weeks | | 1.283 |
| 11 | weeks | | 1.092 |
| 3 | months | 1.720 | |
| 6 | months | 1.541 | |
| 9 | months | 1.375 | |
| 12 | months | 1.192 | |

3) linearity assay of stabilized AST liquid reagent in a single vial stored at 2-8° C.

TABLE 19
stored at 2-8° C. for three months

| | | | | | | |
|---|---|---|---|---|---|---|
| theory value | 0 | 121.7 | 244.1 | 366.0 | 488.0 | 600 |
| U/L | | | | | | |
| assay value U/L | 4.5 | 124 | 247 | 366 | 473 | 582 |

stored at 2-8° C. for six months

| | | | | | | |
|---|---|---|---|---|---|---|
| theory value U/L | 0 | 113 | 226 | 338 | 451 | 564 |
| assay value U/L | 5.0 | 115 | 220 | 338 | 436 | 559 |

stored at 2-8° C. for nine months

| | | | | | | |
|---|---|---|---|---|---|---|
| theory value U/L | 0 | 121 | 222 | 315 | 419 | 548 |
| assay value | 4.6 | 115 | 220 | 315 | 420 | 535 |
| U/L | | | | | | |

stored at 2-8° C. for twelve months

| | | | | | | |
|---|---|---|---|---|---|---|
| theory value U/L | 0 | 114 | 228 | 343 | 457 | 571 |
| assay value | 5.2 | 114 | 231 | 346 | 449 | 548 |
| U/L | | | | | | |

4) accuracy assay of stabilized AST liquid reagent in a single vial stored at 2-8° C.

TABLE 20

| storage at | | |
|---|---|---|
| 2-8° C. | | |
| (months) | Serum I (U/L) | Serum III (U/L) |
| 3 | target value: 28(23-33) | target value: |
| | assay value: 31 | 104(84-124) |
| | | assay value: 105 |
| 6 | target value: 28(23-33) | target value: 104(84-124) |
| | assay value: 29 | assay value: 96 |
| 9 | target value: | target value: 104(84-124) |
| | 28(23-33) | assay value: 100 |
| | assay value: 29 | |
| 12 | target value: | target value: 104(84-124) |
| | 28(23-33) | assay value: 105 |
| | assay value: 28 | |

5) accuracy assay of stabilized AST liquid reagent in a single vial stored at 37° C.

TABLE 21

| | Serum I (U/L) | Serum II (U/L) | Serum III (U/L) |
|---|---|---|---|
| Storage at 37° C. | Target value 30 | Target value 54 | Target value |
| (days) | (20-40) | (41-67) | 101 (81-121) |
| 0 | 28 | 57 | 100 |
| 5 | 30 | 58 | 105 |
| 6 | 29 | 56 | 104 |
| 7 | 27 | 61 | 102 |

The above results showed that the assay data of the stabilized AST reagent was up to the mustard after being stored at 2-8° C. for 12 months or at 37° C. for 7 days. In short, the method of utilizing coenzyme (β-NADH) reduction system of glucose dehydrogenase/D-glucose pair is feasible.

### Example 4

Determination of the stability of ALT reagent (D-glucose: 5 mmol/L, glucose dehydrogenase: 10 U/L) formulated in accordance with the invention is as follows.

One stabilized ALT reagent in a single vial is as follows:

TABLE 22

| | Molecular | Concentration | |
|---|---|---|---|
| Ingredients | Weight | (mmol/L) | /L |
| Tris | 121.1 | | |
| α-ketoglutarate, | 226.1 | | |
| (2H₂O) | | | |
| L-alanine | 89.1 | | |
| D-glucose | 180.2 | | |
| glycerol | 92.1 | | |
| EDTA.2Na | 372.2 | | |
| sodium azide | 65.1 | | |
| β-NADH disodium | 709.4 | | |
| lactate dehydrogenase | | | |
| glucose dehydrogenase | | | |
| hydrochloric acid | 36.5 | | |

The correspondence unstabilized liquid reagent configured as a single vial does not include the coenzyme reduction system of glucose dehydrogenase/D-glucose and absent from glycerol, but comprises the other ingredients as above stable 22.

Storage Conditions:

Sealed and stored 2-8° C. or 37° C.

Spectrophotometric Parameters:

| | |
|---|---|
| wave length: 340 nm | reaction temperature: 37° C. |

cuvette path length: 10 mm
volume ratio of sample to reagent: 1:15

| | |
|---|---|
| lag phase: 60 seconds | reaction time: 60 seconds |

blank absorbance of reagent: >1.0 A
for showing the concentration of NADH
assay accuracy: the result should be
in the range of the quality
controlled serum.
linearity range: ≧550 U/L
assay result:

1) The blank absorbance of ALT liquid reagent in a single vial stored at 37° C.

TABLE 23

| Storage at 37° C. (days) | Stabilized reagent | Unstabilized reagent |
|---|---|---|
| 0 | 1.857 | 1.752 |
| 1 | 1.687 | 1.375 |
| 2 | | 1.066 |
| 3 | | |
| 4 | 1.188 | |
| 5 | 1.057 | |

It is obvious that stabilized ALT reagent has a storage of 5 days at 37° C. But the unstabilized AST reagent has a storage of 2 days at 37° C. β-NADH in the stabilized reagent is more stable than others.

2) The blank absorbance of ALT liquid reagent in a single vial stored at 2-8° C.

TABLE 24

| | Stabilized | |
|---|---|---|
| Storage at 2-8° C. | reagent | Unstabilized reagent |
| 0 week | 1.902 | 1.752 |
| 3 months | 1.688 | 1.235 |
| 4 months | | 1.041 |
| 6 months | 1.468 | |
| 9 months | 1.266 | |
| 12 months | 1.099 | |

It is obvious that the storage of β-NADH in the stabilized ALT reagent is more than 12 months at 2-8° C. But β-NADH in the unstabilized ALT reagent has a storage of 4 months only.

3) linearity assay of stabilized ALT liquid reagent in a single vial stored at 2-8° C.

TABLE 25
stored at 2-8° C. for three months

| | | | | | |
|---|---|---|---|---|---|
| Theory value | 4.3 | 153.4 | 302.5 | 451.5 | 600.6 |
| U/L | | | | | |
| assay value U/L | 4.3 | 164.0 | 315.6 | 477.6 | 600.6 |

stored at 2-8° C. for six months

| | | | | | |
|---|---|---|---|---|---|
| theory value U/L | 8.5 | 177.0 | 345.4 | 513.9 | 682.3 |
| assay value U/L | 8.5 | 199.1 | 367.9 | 541.4 | 682.3 |

stored at 2-8° C. for eight months

| | | | | | |
|---|---|---|---|---|---|
| theory value U/L | 6.7 | 138.1 | 269.5 | 400.9 | 532.3 |
| assay value | 6.7 | 157.8 | 293.2 | 416.5 | 532.3 |
| U/L | | | | | |

stored at 2-8° C. for twelve months

| | | | | | |
|---|---|---|---|---|---|
| theory value U/L | 5.9 | 154.1 | 302.3 | 450.5 | 598.7 |
| assay value | 5.9 | 169.3 | 323.1 | 471.2 | 598.7 |
| U/L | | | | | |

The results of linearity assay of the stabilized ALT liquid reagent in a single vial is up to the mustard after being stored at 2-8° C. for twelve months.

4) accuracy assay of stabilized ALT reagent stored at 37° C.

TABLE 26

| | Serum I (U/L) | Serum II (U/L) | Serum III (U/L) |
|---|---|---|---|
| Storage at 37° C. | Target value 23 | Target value 45 | Target value |
| (days) | (18-28) | (35-55) | 91 (76-106) |
| 0 | 24.3 | 42.8 | 84.5 |
| 3 | 22.1 | 41.8 | 85.6 |
| 5 | 23.8 | 41.4 | 82.0 |

The result of accuracy assay of the stabilized ALT liquid reagent in a single vial are within the target value scope of the quality-controlled serum after being stored at 37° C. for 5 days.

5) accuracy assay of stabilized ALT liquid reagent in a single vial stored at 2-8° C.

TABLE 27

| storage at | | |
|---|---|---|
| 2-8° C. | Serum I (U/L) | Serum III (U/L) |
| (months) | target value: 24(19-29) | target value: 92(77-107) |
| 3 | assay value: 20 | assay value: 82 |
| 6 | assay value: 24 | assay value: 78 |
| 9 | assay value: 24 | assay value: 84 |
| 12 | assay value: 26 | assay value: 88 |

The results of accuracy assay of the stabilized ALT liquid reagent in a single vial are all within the target value scope of the quality controlled serum after being stored at 2-8° C. for 12 months.

The above results showed that the assay data of the stabilized ALT reagent was up to the mustard after being stored at 2-8° C. for 12 months or at 37° C. for 5 days. In short, the method of utilizing coenzyme (NADH) reduction system of glucose dehydrogenase/D-glucose pair is successful.

### Example 5

Determination of the stability of ALT reagent (D-glucose: 1 mmol/L, glucose dehydrogenase: 2 U/L) formulated in accordance with the invention is as follows.

One stabilized ALT liquid reagent in a single vial is as follows:

TABLE 28

| | Molecular | Concentration | |
|---|---|---|---|
| Ingredients | Weight | (mmol/L) | /L |
| tris | 121.1 | | |
| α-ketoglutarate (2H₂O) | 226.1 | | |
| L-alanine | 89.1 | | |
| D-glucose | 180.2 | | |
| glycerol | 92.1 | | |
| EDTA.2Na | 372.2 | | |
| sodium azide | 65.1 | | |
| β-NADH disodium | 709.4 | | |
| lactate dehydrogenase | | | |
| glucose dehydrogenase | | | |
| hydrochloric acid | 36.5 | | |

The correspondence unstabilized liquid reagent configured as a single vial does not include the coenzyme reduction system of glucose dehydrogenase/D-glucose and absent from glycerol, but comprises the ingredients as above stable.

Storage Conditions:

Sealed up stored at 2-8° C. or 37° C.

Spectrophotometric Parameters:

| | |
|---|---|
| wavelength: 340 nm | reaction temperature: 37° C. |

cuvette path length: 10 mm
volume ratio of sample to reagent 1:15

| | |
|---|---|
| lag phase: 60 seconds | reaction time: 60 seconds |

bank absorbance of reagent: >1.0 A
(for showing the concentration of NADH)
assay accuracy: the result should be in
the range of the quality
controlled serum.
Linearity range: ≧550 U/L
assay results:

1) The blank absorbance of ALT reagent stored at 37° C.

TABLE 29

| Storage at 37° C. (days) | Stabilized reagent | Unstabilized reagent |
|---|---|---|
| 0 | 1.710 | 1.705 |
| 1 | 1.525 | 1.330 |
| 2 | | 1.019 |
| 3 | | |
| 4 | 1.016 | |

2) The blank absorbance of ALT reagent stored at 2-8° C.

TABLE 30

| | Stabilized | |
|---|---|---|
| Storage at 2-8° C. | reagent | Unstabilized reagent |
| 0 week | 1.715 | 1.713 |
| 3 months | 1.488 | 1.195 |
| 4 months | | 1.002 |
| 6 months | 1.250 | |
| 9 months | 1.031 | |

3) linearity assay of stabilized ALT liquid reagent in a single vial stored at 2-8° C.

TABLE 31
stored at 2-8° C. for three months

| | | | | | | |
|---|---|---|---|---|---|---|
| theory value | 32 | 129 | 257 | 386 | 514 | 643 |
| U/L | | | | | | |
| assay value U/L | 30 | 128 | 257 | 376 | 507 | 629 |

stored at 2-8° C. for six months

| | | | | | | |
|---|---|---|---|---|---|---|
| theory value U/L | 3.9 | 104 | 208 | 312 | 417 | 520 |
| assay value U/L | 3.9 | 117 | 220 | 312 | 404 | 513 |

Stored at 2-8° C. for nine months

| | | | | | | |
|---|---|---|---|---|---|---|
| theory value U/L | 4.6 | 117 | 234 | 351 | 468 | 565 |
| assay value | 4.6 | 112 | 237 | 351 | 459 | 536 |
| U/L | | | | | | |

4) accuracy assay of stabilized ALT reagent stored at 2-8° C.

TABLE 32

| storage at | | |
|---|---|---|
| 2-8° C. | Serum I (U/L) | Serum III (U/L) |
| (months) | target value: 21(13-29) | target value: 93(73-113) |
| 3 | assay value: 28 | assay value: 90 |
| 6 | assay value: 24 | assay value: 91 |
| 9 | assay value: 22 | assay value: 86 |

5) accuracy assay of stabilized ALT liquid reagent in a single stored at 37° C.

TABLE 33

| | Serum I (U/L) | Serum II (U/L) | Serum III (U/L) |
|---|---|---|---|
| Storage at 37° C. | target value 24 | target value 47 | target value |
| (days) | (19-29) | (37-57) | 92 (77-107) |
| 0 | 22 | 51 | 90 |
| 4 | 28 | 46 | 87 |

The above result showed that the assay data of the stabilized ALT reagent was up to the mustard after being stored at 2-8° C. for 9 months or at 37° C. for 4 days. The method of utilizing coenzyme (NADH) reduction system of glucose dehydrogenase/D-glucose pair is successful.

### Example 6

Determination of the stability of ALT reagent (D-Glucose: 10 mmol/L, Glucose dehydrogenase: 50 U/L) formulated in accordance with the invention is as follows.

One stabilized ALT liquid reagent in a single vial is as follows:

TABLE 34

| | | | |
|---|---|---|---|
| | Molecular | Concentration | |
| ingredients | Weight | (mmol/L) | /L |

| | |
|---|---|
| Tris | 121.1 |
| α-ketoglutarate (2H₂O) | 226.1 |
| L-alanine | 89.1 |
| D-glucose | 180.2 |
| glycerol | 92.1 |
| EDTA.2Na | 372.2 |
| sodium azide | 65.1 |
| β-NADH disodium | 709.4 |
| lactate dehydrogenase | |
| glucose dehydrogenase | |
| hydrochloric acid | 36.5 |

The correspondent unstabilized liquid reagent configured as a single vial does not include the coenzyme reduction system of glucose dehydrogenase/D-glucose and absent from gycerol, but comprises the ingredients as above stable 34.

Storage Conditions:

Sealed up and stored 2-8° C. or 37° C.

Spectrophotometric Parameters:

| | |
|---|---|
| wavelength: 340 nm | reaction temperature: 37° C. |
| cuvette path length: 10 mm | |
| volume ratio of sample to reagent: 1:15 | |
| lag phase: 60 seconds | reaction time: 60 seconds |
| blank absorbance of reagent: >1.0 A | |
| (for showing the concentration of | |
| β-NADH) assay accuracy: assay result | |
| should be in the range of the quality | |
| controlled serum. | |
| Linearity range: ≧550 U/L | |
| assay results: | |

1) The blank absorbance of ALT reagent stored at 37° C.

TABLE 35

| Storage at 37° C. (days) | Stabilized reagent | Unstabilized reagent |
|---|---|---|
| 0 | 1.881 | 1.875 |
| 1 | 1.715 | 1.493 |
| 2 | | 1.154 |
| 3 | | |
| 4 | 1.220 | |
| 5 | 1.092 | |

2) The blank absorbance of ALT reagent stored at 2-8° C.

TABLE 36

| | Stabilized | |
|---|---|---|
| Storage at 2-8° C. | reagent | Unstabilized reagent |
| 0 week | 1.915 | 1.880 |
| 3 months | 1.697 | 1.365 |
| 4 months | | 1.169 |
| 6 months | 1.478 | |
| 9 months | 1.280 | |
| 12 months | 1.115 | |

3) linearity assay of stabilized ALT liquid reagent in a single vial stored at 2-8° C.

TABLE 37
stored at 2-8° C. for three months

| | | | | | |
|---|---|---|---|---|---|
| Theory value | 5.1 | 142 | 284 | 425 | 567 |
| U/L | | | | | |
| assay value U/L | 5.1 | 142 | 294 | 412 | 555 |

stored at 2-8° C. for six months

| | | | | | | |
|---|---|---|---|---|---|---|
| theory value U/L | 4.5 | 125 | 246 | 369 | 492 | 615 |
| assay value U/L | 4.5 | 121 | 244 | 372 | 487 | 604 |

Stored at 2-8° C. for nine months

| | | | | | | |
|---|---|---|---|---|---|---|
| theory value U/L | 5.5 | 80 | 160 | 321 | 481 | 641 |
| assay value | 5.5 | 85 | 170 | 321 | 473 | 625 |
| U/L | | | | | | |

Stored at 2-8° C. for 12 months

| | | | | | | |
|---|---|---|---|---|---|---|
| theory value U/L | 98 | 195 | 293 | 390 | 488 | 580 |
| assay value | 100 | 199 | 287 | 398 | 479 | 561 |
| U/L | | | | | | |

4) accuracy assay of stabilized ALT liquid reagent in a single vial stored at 2-8° C.

TABLE 38

| storage at | | |
|---|---|---|
| 2-8° C. | Serum I (U/L) | Serum III (U/L) |
| (months) | target value: 21(13-29) | target value: 93(73-113) |
| 3 | assay value: 23 | assay value: 84 |
| 6 | assay value: 21 | assay value: 86 |
| 9 | assay value: 26 | assay value: 82 |
| 12 | assay value: 22 | assay value: 87 |

5) accuracy assay of stabilized ALT liquid reagent in a single vial stored at 37° C.

TABLE 39

| | Serum I (U/L) | Serum II (U/L) | Serum III (U/L) |
|---|---|---|---|
| Storage at 37° C. | Target value 24 | Target value 47 | Target value |
| (days) | (19-29) | (37-57) | 92 (77-107) |
| 0 | 21 | 45 | 86 |
| 5 | 23 | 44 | 84 |

The above result showed that the assay data of the stabilized ALT reagent was up to the mustard after being stored at 2-8° C. for 12 months or at 37° C. for 5 days. The method of utilizing coenzyme (β-NADH) reduction system of glucose dehydrogenase/D-glucose pair is successful.

### Example 7

Determination of the stability of UREA reagent (D-glucose: 5 mmol/L, glucose dehydrogenase: 30 U/L) formulated in accordance with the invention is as follows.

One kind of stabilized UREA reagent in a single vial is as follows:

TABLE 40

| | | | |
|---|---|---|---|
| | Molecular | Concentration | |
| ingredients | Weight | (mmol/L) | /L |

| | |
|---|---|
| tris | 121.1 |
| α-ketoglutarate (2H₂O) | 226.1 |
| D-glucose | 180.2 |
| glycerol | 92.1 |
| ADP.K salt | 501.3 |
| sodium azide | 65.1 |
| β-NADH disodium | 709.4 |
| glutamate | |
| dehydrogenase | |
| urease | |
| glucose dehydrogenase | |
| hydrochloric acid | 36.5 |

The correspondent unstabilized liquid reagent configured as a single vial does not include the coenzyme reduction system of glucose dehydrogenase/D-glucose pair and absent from glycerol, but comprises the ingredients as above stable 40.

Storage Conditions:

sealed and stored at 2-8° C. or 37° C.

Spectrophotometric Parameters:

| | |
|---|---|
| wavelength: 340 nm | reaction temperature: 37° C. |
| cuvette path length: 10 mm | |
| volume ratio of sample to reagent 1:100 | |
| lag phase: 30 seconds | |
| reaction time: 60-150 seconds | |
| blank absorbance of reagent: >1.0 A | |
| (for showing the concentration of NADH) | |
| assay precision: assay result should be in | |
| the range of the quality controlled serum. | |
| linearity range: ≧50 mmol/L | |
| assay result: | |

1) The blank absorbance of UREA reagent stored at 37° C.

TABLE 41

| Storage at 37° C. (days) | Stabilized reagent | Unstabilized reagent |
|---|---|---|
| 0 | 1.821 | 1.835 |
| 1 | 1.655 | 1.621 |
| 2 | 1.547 | 1.424 |
| 3 | 1.421 | 1.267 |
| 4 | 1.302 | 1.113 |
| 5 | 1.200 | 0.956 |
| 6 | 1.126 | |
| 7 | 1.051 | |

It is obvious that stabilized UREA reagent has a storage of seven days at 37° C. But the unstabilized UREA reagent has a storage of four days at 37° C. β-NADH in the stabilized reagent is more stable

2) The initial absorbance of UREA reagent stored at 2-8° C.

TABLE 42

| Storage at 2-8° C. | | |
|---|---|---|
| (months) | Stabilized reagent | Unstabilized reagent |
| 0 | 1.773 | 1.835 |
| 3 | 1.611 | 1.519 |
| 6 | 1.507 | 1.271 |
| 8 | | 1.035 |
| 9 | 1.391 | |
| 12 | 1.226 | |
| 15 | 1.125 | |
| 18 | 1.029 | |

The storage of β-NADH in the stabilized UREA reagent is more than 12 months at 2-8° C. But the storage of UREA reagent in the unstabilized is only 8 months at 2-8° C.

3) linearity assay of stabilized UREA reagent stored at 2-8° C.

TABLE 43
stored at 2-8° C. for four months

| | | | | | | |
|---|---|---|---|---|---|---|
| theory value | 1.59 | 10.58 | 21.16 | 31.74 | 42.32 | 52.90 |
| U/L | | | | | | |
| assay value U/L | 1.92 | 11.18 | 22.21 | 31.74 | 43.69 | 52.89 |

stored at 2-8° C. for six months

| | | | | | |
|---|---|---|---|---|---|
| theory value U/L | 1.68 | 14.00 | 28.00 | 42.00 | 56.00 |
| assay value U/L | 1.82 | 14.32 | 28.09 | 41.95 | 54.26 |

stored at 2-8° C. for nine months

| | | | | | |
|---|---|---|---|---|---|
| theory value U/L | 1.62 | 13.50 | 27.00 | 40.50 | 54.00 |
| assay value U/L | 1.80 | 14.14 | 27.55 | 40.16 | 52.08 |

stored at 2-8° C. for twelve months

| | | | | | |
|---|---|---|---|---|---|
| theory value U/L | 1.62 | 13.50 | 27.00 | 40.50 | 54.00 |
| assay value U/L | 1.74 | 14.07 | 27.86 | 39.67 | 51.70 |

stored at 2-8° C. for fifteen months

| | | | | | |
|---|---|---|---|---|---|
| theory value U/L | 1.62 | 13.50 | 27.00 | 40.50 | 54.00 |
| assay value U/L | 1.80 | 13.39 | 27.00 | 38.12 | 50.15 |

stored at 2-8° C. for eighteen months

| | | | | | |
|---|---|---|---|---|---|
| theory value U/L | 1.68 | 14.00 | 28.00 | 42.00 | 56.00 |
| assay value U/L | 1.80 | 14.59 | 28.44 | 40.61 | 53.38 |

The result of linearity assay of the stabilized UREA liquid reagent in a single vial is up to the mustard after being stored at 2-8° C. for eighteen months.

4) accuracy assay of stabilized UREA reagent stored at 37° C.

TABLE 44

| | Serum I (U/L) | Serum II (U/L) | Serum III (U/L) |
|---|---|---|---|
| Storage at 37° C. | Target value 3.0 | Target value | Target value |
| (days) | (2.3-3.7) | 10.2(8.7-11.7) | 18.7(16.5-20.8) |
| 0 | 3.10 | 10.40 | 18.80 |
| 5 | 3.12 | 10.08 | 19.54 |
| 7 | 2.97 | 10.11 | 18.77 |

The results of accuracy assay of the stabilized UREA reagent within the scope of target value of the quality-controlled serum after being stored at 37° C. for seven says.

5) accuracy assay of stabilized UREA reagent stored at 2-8° C.

TABLE 45

| storage at | | |
|---|---|---|
| 2-8° C. | | |
| (months) | Serum I (U/L) | Serum III (U/L) |
| 3 | target value: 2.5(1.8-3.2) | target value: |
| | assay value: 2.76 | 18.5(16.3-20.7) |
| | | assay valu: 18.06 |
| 6 | Target value: 2.5(1.8-3.2) | target value: 18.5(16.3-20.7) |
| | assay value: 2.52 | assay value: 19.40 |
| 9 | Target value: | target value: 18.5(16.3-20.7) |
| | 2.5(1.8-3.2) | assay value: 19.05 |
| | assay value: 2.72 | |
| 12 | Target value: | |
| | 7.70(5.58-9.55) | |
| | assay value: 7.86 | |
| 15 | Target value: | Target value: |
| | 3.0(2.3-3.7) | 18.7(16.5-20.8) |
| | assay value: 2.83 | Assay value: 17.27 |
| 18 | Target value: | Target value: |
| | 3.0(2.3-3.7) | 18.7(16.5-20.8) |
| | assay value: 2.95 | Assay value: 19.05 |

The results of accuracy assay of the stabilized UREA liquid reagent within the scope of target value of the quality-controlled serum after being stored at 2-8 for 12 months.

The above results showed that the assay data of the stabilized UREA reagent was up to the mustard after being stored at 2-8 for eighteen months or at 37 for seven days. In short, the method of utilizing coenzyme (NADH) reduced system of glucose dehydrogenase/D-glucose pair is feasible.

### Example 8

Determination of the stability of UREA reagent (D-Glucose: 1 mmol/L, Glucose dehydrogenase: 5 U/L) formulated in accordance with the invention is as follows.

One stabilized UREA reagent in a single vial is as follows.

Stable 46

| | Molecular | Concentration | |
|---|---|---|---|
| ingredients | Weight | (mmol/L) | /L |
| Tris | 121.1 | | |
| α-ketoglutarate (2H₂O) | 226.1 | | |
| D-glucose | 180.2 | | |
| glycerol | 92.1 | | |
| ADP.K salt | 501.3 | | |
| sodium azide | 65.1 | | |
| β-NADH disodium | 709.4 | | |
| glutamate | | | |
| dehydrogenase | | | |
| urease | | | |
| glucose dehydrogenase | | | |
| hydrochloric acid | 36.5 | | |

The correspondent unstabilized liquid reagent configured as a single vial do not include the coenzyme reduction system of glucose dehydrogenase/D-glucose and absent from glycerol, but comprises the ingredients as above stable 46.

Storage Conditions:

sealed and stored at 2-8 or 37

Spectrophotometric Parameters:

| | |
|---|---|
| wavelength: 340 nm | reaction temperature: 37 |
| cuvette path length: 10 mm | |
| volume ratio of sample to reagent: 1:100 | |
| lag phase: 30 seconds | reaction time: 60-150 seconds |
| blank absorbance of reagent: >1.0 A | |
| (for showing the concentration of NADH) | |
| assay precision: assay results should be in | |
| the range of the quality controlled serum. | |
| linearity range: ≧50 mmol/L | |
| assayed results: | |

2) The blank absorbance of UREA liquid reagent in a single vial stored at 37

TABLE 47

| Storage at 37 □ (days) | Stabilized reagent | Unstabilized reagent |
|---|---|---|
| 0 | 1.550 | 1.557 |
| 1 | 1.379 | 1.345 |
| 2 | 1.265 | 1.114 |
| 3 | 1.130 | 0.986 |
| 4 | 1.008 | |

TABLE 48

| Storage at 2-8 □ | | |
|---|---|---|
| (months) | Stabilized reagent | Unstabilized reagent |
| 0 | 1.562 | 1.555 |
| 3 | 1.393 | 1.240 |
| 6 | 1.285 | 0.996 |
| 9 | 1.163 | |
| 12 | 1.001 | |

3) linearity assay of stabilized UREA reagent stored at 2-8

TABLE 49
stored at 2-8° C. for three months

| | | | | | |
|---|---|---|---|---|---|
| theory value U/L | 1.70 | 12.80 | 25.60 | 38.40 | 51.20 |
| assay value U/L | 1.80 | 13.39 | 27.00 | 38.12 | 50.15 |

stored at 2-8° C. for six months

| | | | | | |
|---|---|---|---|---|---|
| theory value U/L | 10.50 | 21.00 | 31.50 | 42.00 | 52.50 |
| assay value U/L | 11.36 | 22.11 | 31.75 | 41.61 | 52.69 |

stored at 2-8° C. for nine months

| | | | | | |
|---|---|---|---|---|---|
| theory value U/L | 10.50 | 21.00 | 31.50 | 42.00 | 52.50 |
| assay value U/L | 11.12 | 21.84 | 31.61 | 41.50 | 50.18 |

stored at 2-8° C. for twelve months

| | | | | | |
|---|---|---|---|---|---|
| theory value U/L | 10.22 | 20.44 | 30.66 | 40.88 | 51.10 |
| assay value U/L | 10.80 | 20.50 | 29.77 | 39.14 | 48.75 |

4) accuracy assay of stabilized UREA reagent stored at 37

TABLE 50

| | Serum I (U/L) | Serum II (U/L) | Serum III (U/L) |
|---|---|---|---|
| Storage at 37 | Target value | Target value | Target value |
| □ (days) | 2.5(1.8-3.2) | 9.0(7.5-10.5) | 18.5(16.3-20.7) |
| 0 | 2.67 | 9.83 | 18.14 |
| 4 | 2.85 | 9.77 | 18.50 |

5) accuracy assay of stabilized UREA reagent stored at 2-8

TABLE 51

| storage at | Serum I (U/L) | Serum III (U/L) |
|---|---|---|
| 2-8 □ | target value: | target value: |
| (months) | 2.7(2.0-3.3) | 18.7(16.5-20.8) |
| 3 | 2.91 | 19.20 |
| 6 | 3.00 | 19.01 |
| 9 | 2.98 | 18.90 |
| 12 | 2.90 | 19.00 |

The above results showed that the assay data of the stabilized UREA reagent was up to the mustard after being stored at 2-8 for twelve months or at 37 for four days. In short, the method of utilizing coenzyme (NADH) reduction system of glucose dehydrogenase/D-glucose pair is feasible.

### Example 9

Determination of the stability of UREA reagent (D-glucose: 10 mmol/L, glucose dehydrogenase: 50 U/L) formulated in accordance with the invention is as follows.

One kind of stabilized UREA reagent in a single vial is as follows:

TABLE 52

| | Molecular | Concentration | |
|---|---|---|---|
| ingredients | Weight | (mmol/L) | /L |
| tris | 121.1 | | |
| α-ketoglutarate (2H₂O) | 226.1 | | |
| D-glucose | 180.2 | | |
| glycerol | 92.1 | | |
| ADP.K salt | 501.3 | | |
| sodium azide | 65.1 | | |
| β-NADH disodium | 709.4 | | |
| glutamate | | | |
| dehydrogenase | | | |
| urease | | | |
| glucose dehydrogenase | | | |
| hydrochloric acid | 36.5 | | |

The correspondent unstabilized liquid reagent configured as a single vial does not include the coenzyme reduced system of glucose dehydrogenase/D-glucose and absent from glycerol, but comprises the ingredients as above stable.

Storage Conditions:

sealed and stored at 2-8 or 37

Spectrophotometric Parameters:

| | |
|---|---|
| wavelength: 340 nm | reaction temperature: 37 |
| cuvette path length: 10 mm | |
| volume ratio of sample to reagent: 1:100 | |
| lag phase: 30 seconds | reaction time: 60-150 seconds |
| blank absorbance of reagent: >1.0 A | |
| (for showing the concentration of NADH) | |
| assay precision: assay results should be | |
| in the range of the quality | |
| controlled serum. | |
| Linearity range: ≧50 mmol/L | |
| assay results: | |

3) The blank absorbance of UREA reagent stored at 37

TABLE 53

| Storage at 37 □ (days) | Stabilized reagent | Unstabilized reagent |
|---|---|---|
| 0 | 1.942 | 1.938 |
| 1 | 1.780 | 1.723 |
| 2 | 1.675 | 1.520 |
| 3 | 1.553 | 1.362 |
| 4 | 1.440 | 1.205 |
| 5 | 1.341 | 1.047 |
| 6 | 1.273 | |
| 7 | 1.205 | |

2) The blank absorbance of UREA liquid reagent in a single vial stored at 2-8

TABLE 54

| Storage at 2-8 □ | | Unstabilized |
|---|---|---|
| (months) | Stabilized reagent | reagent |
| 0 | 1.933 | 1.930 |
| 3 | 1.770 | 1.613 |
| 6 | 1.669 | 1.362 |
| 8 | | 1.124 |
| 9 | 1.555 | |
| 12 | 1.392 | |
| 15 | 1.295 | |
| 18 | 1.203 | |

3) Linearity assay of stabilized UREA reagent stored at 2-8

TABLE 55
stored at 2-8 [symbol] for three months

| | | | | | |
|---|---|---|---|---|---|
| theory value U/L | 11.47 | 22.93 | 34.40 | 45.87 | 57.34 |
| assay value U/L | 11.96 | 23.02 | 34.40 | 43.62 | 55.43 |

stored at 2-8 [symbol] for six months

| | | | | | |
|---|---|---|---|---|---|
| theory value U/L | 10.03 | 20.06 | 30.09 | 40.12 | 50.15 |
| assay value U/L | 10.03 | 20.80 | 31.43 | 40.12 | 50.29 |

stored at 2-8 [symbol] for nine months

| | | | | | |
|---|---|---|---|---|---|
| theory value U/L | 11.30 | 22.60 | 34.00 | 45.30 | 56.60 |
| assay value U/L | 11.90 | 23.20 | 34.00 | 43.70 | 54.20 |

stored at 2-8 [symbol] for twelve months

| | | | | | |
|---|---|---|---|---|---|
| theory value U/L | 11.66 | 23.31 | 34.97 | 46.63 | 58.29 |
| assay value U/L | 12.07 | 24.44 | 34.97 | 46.22 | 56.96 |

stored at 2-8 [symbol] for fifteen months

| | | | | | |
|---|---|---|---|---|---|
| theory value U/L | 11.00 | 21.90 | 32.90 | 43.80 | 54.80 |
| assay value U/L | 11.80 | 22.40 | 31.70 | 43.80 | 52.30 |

stored at 2-8 [symbol] for eighteen months

| | | | | | |
|---|---|---|---|---|---|
| theory value U/L | 10.27 | 20.54 | 30.80 | 41.07 | 51.34 |
| assay value U/L | 11.09 | 21.73 | 31.95 | 41.07 | 50.81 |

4) The accuracy assay of stabilized UREA reagent stored at 37

TABLE 56

| Storage | Serum I (U/L) | Serum II (U/L) | Serum III (U/L) |
|---|---|---|---|
| at 37 □ | target value | target value | target value |
| (days) | 2.5(1.8-3.2) | 9.0(7.5-10.5) | 18.5(16.3-20.7) |
| 0 | 2.64 | 9.17 | 18.64 |
| 4 | 2.51 | 9.70 | 18.73 |
| 7 | 2.47 | 9.64 | 19.20 |

5) Accuracy assay of stabilized UREA liquid reagent stored at 2-8

TABLE 57

| storage at | Serum I (U/L) | Serum III (U/L) |
|---|---|---|
| 2-8 □ | target value: | target value: |
| (months) | 2.7(2.0-3.3) | 18.7(16.5-20.8) |
| 3 | 2.77 | 18.35 |
| 6 | 2.80 | 18.86 |
| 9 | 2.90 | 18.65 |
| 12 | 2.92 | 18.60 |
| 15 | 2.98 | 19.00 |
| 18 | 3.00 | 18.73 |

The above result showed that the assay data of the stabilized UREA reagent were up to the mustard after being stored at 2-8 for eighteen months or at 37 for seven days. In short, the method of utilizing coenzyme (NADH) reduced system of glucose dehydrogenase/D-glucose pair is feasible.

### INDUSTRY USABILITY

In this invention, because of utilization of an antioxidant coenzyme reduction system which comprises highly specific enzyme/substrate pair, so the amounts of enzyme and substrate are reduced greatly, and the cost of reagent does not increase almost. Moreover, the stability of the reagent is enhanced, which resulted from avoiding introduction of other enzymes along with the massive stable enzymes addition.

1. A reagent for the determination of an analyte concentration in a patient wherein the degree of oxidation rate of a coenzyme is measured, that said reagent is stabilized against oxidation by a coenzyme reduction system comprising a special enzyme and a substrate pair selected so as to enable continuous regeneration of said coenzyme throughout storage of said reagent, characterized in that said reagent comprising an enzyme with complete specificity for said substrate.

2. The reagent of claim 1 which is configured as a single vial.

3. The reagent of claim 1 wherein said enzyme/substrate pair is glucose dehydrogenase/D-glucose.

4. The reagent of claim 3 wherein said analyte is aspartate transaminase.

5. The reagent of claim 4 wherein said glucose dehydrogenase in the range of 2-100 U/L, said D-glucose from 0.1 to 20 mmol/l.

6. The reagent of claim 5 wherein said glucose dehydrogenase in the range of 5-50 U/L, said D-glucose from 1 to 10 mmol/l.

7. The reagent of claim 3 wherein said analyte is alanine transaminase.

8. The reagent of claim 7 wherein said glucose dehydrogenase is in the range of 2-100 U/L, said D-glucose from 0.1 to 20 mmol/l.

9. The reagent of claim 8 wherein said glucose dehydrogenase is in the range of 2-50 U/L, said D-glucose from 1 to 10 mmol/l.

10. The reagent of claim 3 wherein said analyte is blood urea.

11. The reagent of claim 10 wherein said glucose dehydrogenase is in the range of 2-100 U/L, said D-glucose from 1 to 10 mmol/l.

12. The reagent of claim 11 wherein said glucose dehydrogenase is in the range of 5-50 U/L, said D-glucose from 1 to 10 mmol/l.

13. The reagent of claim 2 wherein said enzyme/substrate pair is glucose dehydrogenase/D-glucose.
